(19) United States
(12) Patent Application Publication
(10) Pub. No.: US 2016/0338682 A1
(43) Pub. Date: Nov. 24, 2016

(54) PNEUMATIC SYSTEM AND METHOD FOR INTERMITTENTLY RIGIDIFYING AN ENDOSCOPIC SPECIMEN RETAINING CARRIER

(71) Applicants: Lennox Hoyte, Tampa, FL (US); Anthony Nosa Imudia, Lutz, FL (US)

(72) Inventors: Lennox Hoyte, Tampa, FL (US); Anthony Nosa Imudia, Lutz, FL (US)

(73) Assignee: University of South Florida, Tampa, FL (US)

(21) Appl. No.: 15/162,011

(22) Filed: May 23, 2016

Related U.S. Application Data
(60) Provisional application No. 62/165,632, filed on May 22, 2015.

(51) Int. Cl. A61B 17/00; A61B 1/313; A61B 17/34

(52) U.S. Cl. CPC A61B 17/00234; A61B 17/3474; A61B 1/3132; A61B 17/3423; A61B 2017/00287; A61B 2017/00544; A61B 2217/005; A61B 2017/320024

(57) ABSTRACT

An insufflation/desufflation apparatus/system, and method of use thereof. An airtight tube is secured circumferentially to the opening of an endoscopic containment bag. The tube is attached to a valve and a small conduit for insufflating (pressurizing) and desufflating (depressurizing) the tube. The tube is formed of a highly pliable material, similar to the plastic of the pliable containment bag. When the tube is desufflated, the bag and tube are easily folded and wrapped for passage into the peritoneal cavity via a standard laparoscopic port. When the tube is insufflated via the conduit, air fills the closed tube and makes the whole structure substantially rigid in the shape of the containment bag opening around which it is secured. The result is that the bag is forced open by the now rigid, insufflated tube.

### CROSS-REFERENCE TO RELATED APPLICATIONS

This nonprovisional application claims priority to U.S. Provisional Patent Application No. 62/165,632, entitled “Pneumatic Method for Intermittently Rigidifying an Endoscopic Specimen Containment Bag”, filed May 22, 2015, which is incorporated herein by reference in its entirety.

### BACKGROUND OF THE INVENTION

1. Field of the Invention

This invention relates, generally, to endoscopic containment bags. More specifically, it relates to pneumatic systems and methodologies applied to such containment bags during endoscopic surgical procedures.

2. Brief Description of the Prior Art

Since the introduction of minimally invasive gynecologic surgeries in the late 1990s, millions of patients have benefited enormously from this technological advancement. Minimally invasive hysterectomy and myomectomy through either traditional laparoscopy or robotic assistance has been possible due to the use of open mechanical power morcellation as a means of retrieving the surgical specimen.

While this open mechanical power morcellation has been advantageous in facilitating these complex surgeries, it has the disadvantage of potentially spreading previously undiagnosed uterine malignancy during the process. Recently, the Food & Drug Administration (“FDA”) issued a safety communication discouraging the use of open laparoscopic power morcellation for the surgical removal of the uterus following hysterectomy or uterine fibroids following myomectomy in women. This safety communication was issued because this type of procedure poses the risk of disseminating unsuspected malignant tissue, such as uterine sarcomas. To continue to harness the multiple benefits of minimally invasive gynecologic surgeries, it is imperative that laparoscopic surgeons devise a safe alternative to current open power morcellation.

When a conventional endoscopic containment bag is introduced into the body cavity, it must be rolled up tightly in order to pass through a narrow (5-10 millimeter diameter) endoscopic port. Once inside the cavity, the bag has to be opened up in order to place the specimen for extraction. The process of opening up the bag and placing the specimen can be tedious and unpredictable using the endoscopic instruments.

Accordingly, what is needed is an apparatus and/or methodology for maintaining an endoscopic containment bag in an open position in a body cavity, which would in turn make specimen insertion easier and predictable, saving time and frustration. However, in view of the art considered as a whole at the time the present invention was made, it was not obvious to those of ordinary skill in the field of this invention how the shortcomings of the prior art could be overcome.

While certain aspects of conventional technologies have been discussed to facilitate disclosure of the invention, Applicants in no way disclaim these technical aspects, and it is contemplated that the claimed invention may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### BRIEF SUMMARY OF THE INVENTION

The long-standing but heretofore unfulfilled need for an endoscopic apparatus or system that facilitates placement of an excised tissue specimen therewithin is now met by a new, useful, and nonobvious invention.

In an embodiment, the current invention is a pneumatic system for use with a pliable endoscopic retaining carrier that has a specimen-receiving opening and a substantially hollow interior, where the retaining carrier is insertable into an operative internal cavity (e.g., abdominal or pelvic cavity) of a subject or patient. The pneumatic system includes a pneumatic base channel disposed along a perimeter of the carrier's specimen-receiving opening and a plurality of pneumatic side channels disposed at a spaced distance away from each other along the carrier's outer surface. The pneumatic channels are interconnected and have substantially hollow interiors that are in open communication with each other. The system further includes a filling channel in communication with at least one of the pneumatic channels (e.g., directly coupled to the pneumatic base channel). The filling channel is also configured to be coupled to a fluid source for pumping a fluid into the filling channel and thus also into the interiors of the pneumatic channels.

The pneumatic channels each have a desufflated position and an insufflated position; similarly, the retaining carrier has a collapsed position and a rigidified position that respectively correspond to the pneumatic channels' desufflated position and insufflated position during. Desufflated pneumatic channels and collapsed retaining carrier generally occurs during insertion and withdrawal of the retaining carrier, including the pneumatic channels, into and out of the operative internal cavity of the subject or patient. Insufflated pneumatic channels and expanded retaining carrier generally occurs during placement of an excised specimen within the patient's internal operative cavity into the interior of the retaining carrier. Insufflation of the pneumatic channels rigidifies the retaining carrier, and desufflation of the pneumatic channels collapses the retaining carrier.

The pneumatic system may further include a valve disposed on and in communication with the filling channel, where the valve is configured to be coupled to the fluid source for controlling fluid flow into the filling channel and into the pneumatic channels.

The pneumatic side channels may be disposed substantially equidistant from each other around the retaining carrier. Further, the pneumatic side channels can extend along a length of the retaining carrier and converge/interconnect at an apex of the retaining carrier opposite from the center of the specimen-receiving opening.

In a separate embodiment, the current invention is an endoscopic apparatus that includes not only the pneumatic system as discussed above but also the retaining carrier discussed above. Referring specifically to the retaining carrier now, the carrier may include a means of tightening, cinching, closing, or sealing the specimen-receiving opening positioned along the perimeter of the opening. Optionally, this means may be a drawstring-type apparatus that is pulled relative to the opening in order to reduce a diameter or length of the opening. Alternatively, the specimen-receiving opening can be retrieved through one of the ports and tightened against the trocar to maintain pneumoperitoneum. Additionally, the insufflated carrier may be pressed up against the anterior abdominal wall within the patient.

The carrier may also include a plurality of elongate, flexible laparoscopic tool- or trocar-receiving channels that extend externally from an outer surface of the retaining bag at a spaced distance away from the specimen-receiving opening, at a spaced distance away from each other, and at a spaced distance away from the pneumatic channels. The laparoscopic tool- or trocar-receiving channels are positioned on the side of the bag, such that the channels line up with the laparoscopic ports on a body of the patient. The channels further are structured to receive one or more laparoscopic tools. Each channel has a proximal end and a distal end, where the distal end terminates at the body of the retaining bag within the operative internal cavity of the patient and the proximal end is external to the body of the patient. Each channel also has a substantially hollow interior that is in communication with the hollow interior of the retaining bag. The retaining bag has a first position and a second position. The first position is the bag in a desufflated position within the operative internal cavity of the patient with the specimen-receiving opening being open. The second position is the bag in an insufflated position within the abdominal or pelvic cavity of the patient with the specimen-receiving opening being closed or cinched.

The channels may include a morcellator channel receiving a morcellator, a camera channel receiving a channel, and a control instrument channel receiving a control instrument, such that the morcellator could morcellate the specimen within the interior of the bag. In this case, the morcellator can morcellator the targeted specimen within the retaining bag under direct visualization of the camera while the control instrument holds the specimen.

In a separate embodiment, the current invention is a method of performing a minimally invasive laparoscopic surgery on a subject or patient using the pneumatic system according to certain embodiments discussed above and a retaining carrier according to certain embodiments discussed above. Laparoscopic ports are formed on the body of the patient according to known methods. A targeted specimen (e.g., uterus) is excised within the patient's internal operative cavity (e.g., abdominal or pelvic cavity) according to known methods. A pneumatic morcellation system is inserted into the operative cavity in a deflated position. The morcellation system includes a pliable retaining carrier having a substantially hollow interior and a closeable or sealable specimen-receiving opening, where the opening provides for completely open communication between the patient's operative internal cavity and the carrier's interior. The morcellation system further includes a pneumatic base channel disposed along a perimeter of the specimen-receiving opening, a plurality of pneumatic side openings disposed along an outer surface of the retaining carrier, and a filling channel in communication with at least one of the pneumatic channels. After this is all inserted into the patient, the pneumatic channels are insufflated through the filling channel to expand or rigidify the retaining carrier, and the excised specimen is placed within the interior of the carrier through the specimen-receiving opening.

The pneumatic portion of the morcellation system (i.e., base channel, side channels, and filling channel) may be retrofitted onto an existing retaining carrier, or it may be integrated with the retaining carrier. If the former, these channels would be attached to the retaining carrier prior to inserting the morcellation system into the patient's operative internal cavity. Specifically, the pneumatic base channel would be attached along the perimeter of the specimen-receiving opening, and the pneumatic side channels would be attached along the outer surface of the retaining carrier.

If the morcellation system does include the laparoscopic tool- or trocar-receiving channels as discussed above, each of these channels can be withdrawn from the patient's cavity through their respective laparoscopic ports. Optionally, a suture tag disposed on each of these channels can be used to facilitate manipulation of the channels. The specimen-receiving opening is tightened, cinched, closed, or sealed to enclose the specimen within the carrier (e.g., pulling a drawstring-type apparatus to reduce a diameter or length of the opening), and optionally, the cinched opening can be positioned against an anterior wall of the patient's operative internal cavity. One or more laparoscopic tools can be inserted into one of these channels, such that one end is in the retaining carrier's interior and the opposite end is outside the patient. The retaining carrier can then be insufflated to distend in order to form a protected environment. The specimen can then he morcellated and the pieces withdrawn from carrier out of the patient's body. When a sufficient amount of the specimen has been removed, the retaining carrier can be desufflated, and the morcellation system can be withdrawn from the patient's body through any of the laparoscopic ports, while the retaining carrier would continue to enclose any leftover remnants of the morcellated specimen.

Optionally, before withdrawing the laparoscopic- or trocar-receiving channels from the patient's body through the laparoscopic ports, the pneumatic channels can be desufflated. This can facilitate the laparoscopic- or trocar-receiving channels fitting through the laparoscopic ports and can help reduce chances of damage or breakage if forcing the laparoscopic- or trocar-receiving channels fitting through the laparoscopic ports. In a further embodiment, the pneumatic channels can be re-insufflated after the laparoscopic- or trocar-receiving channels have been withdrawn through the laparoscopic ports. This can help further rigidify the retaining carrier to form the protected environment.

In an embodiment, the step of withdrawing each channel from the operative cavity can be performed as follows. A grasper can be inserted into a first laparoscopic port and withdrawing a first channel. This can be repeated for withdrawing a second channel from a second laparoscopic port. In this case, the step of tightening the specimen-receiving opening is performed after withdrawing the first channel but before withdrawing the second channel.

In an embodiment, the step of withdrawing or removing the morcellation system from the operative cavity can be performed as follows. Each channel, except for one (1), can be inserted back into the operative cavity after the laparoscopic tools have been removed. The remaining channel can then be pulled in order to extract the retaining carrier and each channel from the operative cavity.

Each of the foregoing steps may be performed under direct visualization of a camera.

These and other important objects, advantages, and features of the invention will become clear as this disclosure proceeds.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

For a fuller understanding of the invention, reference should be made to the following detailed description, taken in connection with the accompanying drawings, in which:

FIG. 1 depicts a structure of a power morcellation system including pneumatic channels, according to an embodiment of the current invention.

FIG. 2A depicts a containment bag within a subject's body with pneumatic channels desufflated.

FIG. 2B depicts the containment bag of FIG. 2A but with pneumatic channels insufflated and an excised specimen positioned within the containment bag.

FIG. 2C depicts the containment bag of FIG. 2B with the specimen positioned within the containment bag but with the pneumatic channels desufflated in preparation for the remainder of the endoscopic procedure.

FIG. 3A is a cross-sectional view of a subject's body with a user-operated grasper grasping a channel within the cavity.

FIG. 3B is a cross-sectional view of the subject's body with the channel of FIG. 3A pulled/withdrawn through a laparoscopic port via the user-operated grasper.

FIG. 3C is a cross-sectional view of the subject's body with four channels each pulled/withdrawn through a respective laparoscopic port.

FIG. 3D is a top view of the subject's body with four channels each pulled/withdrawn through a respective laparoscopic port. Overall, FIGS. 3A-3D depict withdrawal of the channels from the cavity, along with depicting all four channels extending out of the cavity but without any laparoscopic tools inserted therein.

FIG. 4A is a cross-sectional view of a portion of a user's pelvic cavity with an open retaining bag/carrier contained therein and with laparoscopic tools inserted into the channels of the system and into the interior of the retaining bag. A grasper can also be seen attempting to grasp a drawstring for tightening or closing the opening that receives the specimen to be removed.

FIG. 4B depicts the positioning of FIG. 4A except with the grasper tightening the drawstring in order to tighten or close the opening that receives the specimen to be removed. As such, the specimen can be sealed within the interior of the retaining bag.

FIG. 5 is a view within an insufflated retaining bag through a laparoscopic camera, showing the control instrument and morcellator during morcellation of the blue specimen.

FIG. 6A depict channels extending out of the subject's body with laparoscopic tools (other than the camera) removed.

FIG. 6B depicts removal of the camera and two of the channels being inserted back into the abdominal/pelvic cavity of the subject.

FIG. 6C depicts three of the channels inserted into the abdominal/pelvic cavity of the subject with the morcellator channel still extending out of the subject's body.

FIG. 6D depicts removal of the morcellation system from the subject's body through the morcellation port. Overall, FIGS. 6A-6D depict a process of removal of the morcellation system from the subject's body.

FIG. 7 depicts remnants of the specimen remaining contained within the retaining bag even after morcellation and removal of the retaining bag from the abdominal/pelvic cavity of the subject or patient.

### DETAILED DESCRIPTION OF THE PREFERRED EMBODIMENT

In the following detailed description of the preferred embodiments, reference is made to the accompanying drawings, which form a part thereof and within which are shown by way of illustration specific embodiments by which the invention may be practiced. It is to be understood that other embodiments may be utilized and structural changes may be made without departing from the scope of the invention.

As used in this specification and the appended claims, the singular forms “a”, “an”, and “the” include plural referents unless the content clearly dictates otherwise. As used in this specification and the appended claims, the term “or” is generally employed in its sense including “and/or” unless the context clearly dictates otherwise.

In an embodiment, the current invention allows a pliable containment bag—such as that seen in U.S. Pat. No. 9,044,210 to the current inventors—to be rolled and/or folded and passed into an endoscopic surgical field via normal-sized laparoscopic trocar ports. The rolled/folded containment bag can then be opened up by insufflating a plurality of pneumatic channels disposed on the bag, thus allowing the bag to unroll and/or open up itself within the patient or subject's body. Typically, a pneumatic channel is positioned along the perimeter of the opening into the interior of the containment bag, and a plurality of pneumatic channels are dispose along the sides of the bag with one end of each of these side channels terminating at the channel disposed along the perimeter of the bag opening. All of these pneumatic channels can be interconnected, such that insufflating one of these channels would insufflate all of the channels, thus “rigidifying” the containment bag.

Insufflating or rigidifying the bag in such a manner provides an easier mechanism for opening/expanding the bag than trying to open it and keeping it open using laparoscopic instruments, while attempting to place the excised specimen into the bag. It is contemplated herein that this pneumatic system can be utilized by endoscopic surgeons for a range of endoscopic tissue extractions, for example including, but not limited to, uteruses gallbladders, prostates, and portions of small and large bowel, among other tissue masses, all of which can be performed laparoscopically and/or robotically.

Methodologically, an airtight tube is secured circumferentially to the opening of a conventional endoscopic containment bag or even the containment bag of U.S. Pat. No. 9,044,210. The tube is attached to a valve and a small conduit for insufflating (pressurizing) and desufflating (depressurizing) the tube with air or other fluid. The tube is formed of a highly pliable material, for example similar to the plastic of the pliable containment bag.

When the tube is desufflated, the bag and tube are easily folded and wrapped for passage into the peritoneal cavity via a standard laparoscopic port. When the tube is insufflated via the conduit, air fills the closed tube and makes the whole structure substantially rigid in the shape of the containment bag opening around which it is secured. The result is that the bag is forced open by the now rigid, insufflated tube.

The endoscopic specimen may now be easily placed into the opening of the containment hag for retrieval. Once the specimen is in the bag, the tube can be desufflated, such that the entire bag again becomes extremely pliable for endoscopic extraction or other purposes, such as morcellation.

Similar tubes can also be secured along the outer depth of the containment bag, coursing either circumferentially or from the mouth to the apex of the bag, or a combination thereof. When these tubes are insufflated, walls of the bag are rigidified, and the entire bag can expand to accommodate the specimen. When desufflated, the entire bag becomes pliable.

### EXAMPLE 1

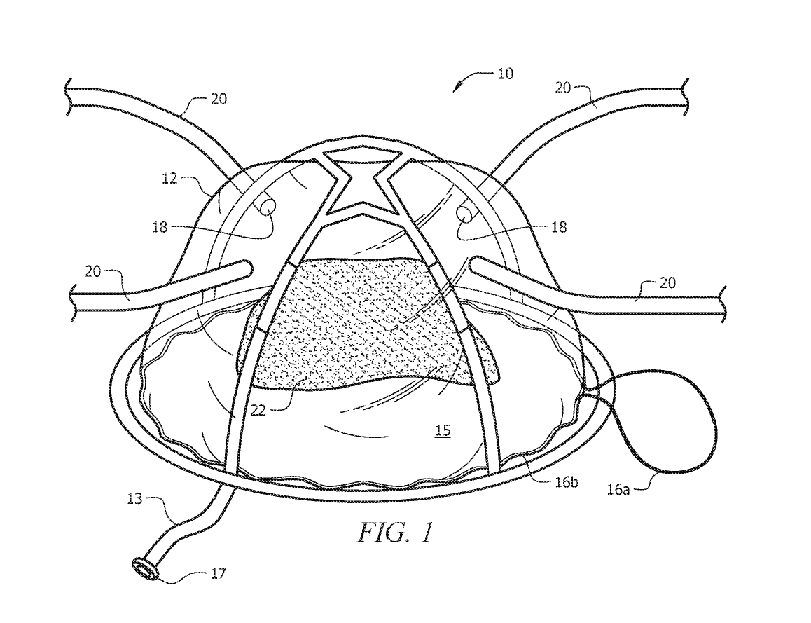

In an embodiment, as seen in FIG. 1, the current invention is a power morcellation system, generally denoted by the reference numeral 10, that includes a sturdy, pliable (e.g., able to be inserted and retracted through a ˜10-15 mm morcellator port), distensible, waterproof/watertight/water-resistant retaining bag/pouch/carrier, denoted by the reference numeral 12, to be deployed into an operative or targeted (e.g., pelvic, abdominal, peritoneal) cavity of a subject or patient. Carrier 12 also includes large base opening 14 surrounded by a tightening or cinching means, such as elastic drawstring 16a, 16b, for receiving targeted, excised specimen 22 within carrier 12.

Large base opening 14 is lined with pneumatic channel 11a. Further, an array of pneumatic channels, denoted by the reference numeral 11b, is positioned along the surface of the wall of retaining carrier 12. All channels 11a, 11b would be in open communication with each other with filling channel 13 extending from one of pneumatic channels 11a, 11b. It is not significant as to which of channels 11a, 11b has filling channel 13 disposed thereon. Valve 17 may be disposed on filling channel 13 for coupling to a conventional air or fluid source (not shown). Once the air or fluid source is coupled to filling channel 13 via filling channel valve 17, a fluid is pumped or otherwise inserted into pneumatic channels 11a, 11b. This allows pneumatic channels 11a, 11b to expand outwardly, thus allowing the walls of retaining carrier 12 to expand or “rigidify” and open interior 15 of carrier 12 for placement of specimen 22 therein.

Pneumatic channels 11a, 11b, along with filling channel 13 and optional valve 17 form the pneumatic system according to the current invention. Certain embodiments of the current invention are the pneumatic system itself, as may be retrofitted or otherwise applied onto an existing containment bag, where pneumatic channel 11a would be affixed to a perimeter of the large opening of the conventional containment hag (e.g., large opening 14 of carrier 12) and pneumatic channels 11b would be affixed to the sides of the conventional containment bag (e.g., sides of carrier 12). In other embodiments, the current invention can be the pneumatic systems integrated with an endoscopic specimen containment bag. An example of such an endoscopic specimen containment bag is carrier 12 in FIG. 1 to form power morcellation system 10.

Still referring to FIG. 1, considering pneumatic channels 11a, 11b being used with carrier 12, carrier 12 can have a plurality of apertures 18 positioned at a spaced distance away from opening 14. Aligned with and extending from apertures 18 is a plurality (e.g., at least three (3) or four (4)) of port tube channels 20 extending outwardly from carrier 12, wherein the interior of each channel 20 is in communication with interior 15 of carrier 12. Each channel 20 has an open proximal end (opposite from the distal end that terminates in apertures 18 of carrier 12) through which a laparoscopic/robotic camera and other instruments (e.g., camera, control instruments, morcellator, etc. may pass.

Methodologically, upon properly creating laparoscopic ports leading to the targeted cavity of the subject according to conventional methods, carrier 12 of morcellation system 10 is brought into the endoscopic field after specimen 22 to be removed is separated from the surrounding tissue. Pneumatic channels 11a, 11b are insufflated via filling channel 13 and valve 17 (e.g., using insufflation source 19), thus rigidifying carrier 12. Specimen 22 is placed into interior 15 of carrier 12 via large opening 14 of carrier 12, and pneumatic channels 11a, 11b are desufflated with specimen 22 remaining within interior 15 of carrier 12. This desufflation can be performed also using insufflation source 19. Before or after desufflation of pneumatic channels 11a, 11b, drawstring 16a (or other tightening or cinching mechanism) is pulled or otherwise actuated to tighten surrounding drawstring 16b (or other corresponding tightening or cinching mechanism) so that large opening 14 is cinched or closed. Drawstring 16a can optionally be retrieved using an endoscopic CARTER-THOMPSON type retriever needle, passed transabdominally. The neck of the cinched large opening can be held against the anterior abdominal wall, so as to restrict the escape of fluid from the cinched large opening.

Port channels 20 are individually pulled back through the laparoscopic ports (e.g., camera port, morcellator port, control instrument port) that lead to the targeted or operative cavity.

As will become clearer as this specification continues, a smaller tube channel can also extend outwardly from carrier 12 and can be suited as an insufflation port channel, among other uses. The smaller port channel can be brought out through a smaller (e.g., 3 mm) trocar port and can be used to insufflate carrier 12. The camera, standard morcellator, and control instruments can then be maintained in or reinserted into carrier 12, which is inside the operative abdominal) cavity. Carrier 12 is insufflated via the insufflation channel, such that it distends to line the peritoneal cavity. Carrier 12 may be able to withstand insufflation pressures of ˜20-40 hhMg. As an additional or alternative option, pneumatic channels 11a, 11b may be insufflated to rigidify/expand carrier 12 to facilitate any procedures to be performed on specimen 22 within interior 15 of carrier 12.

The morcellation is conducted in a typical manner, under direct visualization, with the distal end of the camera and control instrument positioned within interior 15 of insufflated carrier 12, along with the standard morcellator and extraction instrument. If extraneous tissue pieces of specimen 22 break free during the morcellation, these are automatically retained within interior 15 of carrier 12.

When the larger pieces of targeted specimen 22 are removed via the morcellator, carrier 12 can be suctioned to retrieve the smaller remnants, and the instruments can be removed from ports 20. Port channels 20 can then be tied off, pneumatic channels 11a, 11b may be desufflated (if they were insufflated prior to morcellation), and carrier 12 (which would now be substantially empty) can be withdrawn sealed and intact via one of ports 20, for example the larger morcellator port. Laparoscopic port extraction and closure can be performed in a standard fashion.

In an embodiment, the current invention facilitates expansion of a retaining bag or carrier, primarily for placement of the excised specimen therein but potentially also for any procedures to take place therein. All tissue pieces can be kept inside a sealed cavity, eliminating the risk of tissue dispersal into the peritoneal cavity. Morcellation can be accomplished under direct visualization, while the targeted tissue is sealed within the retaining bag or carrier. Normal, healthy, or non-targeted tissue can then be kept or maintained outside of the field of morcellation. The current methodology is user-friendly for deployment and extraction of the device and contents, and standard laparoscopic/robotic equipment can be used. The invention requires only deployment of the carrier and thus fits into the existing laparoscopic workflow at a minimal manufacturing cost.

### EXAMPLE 2

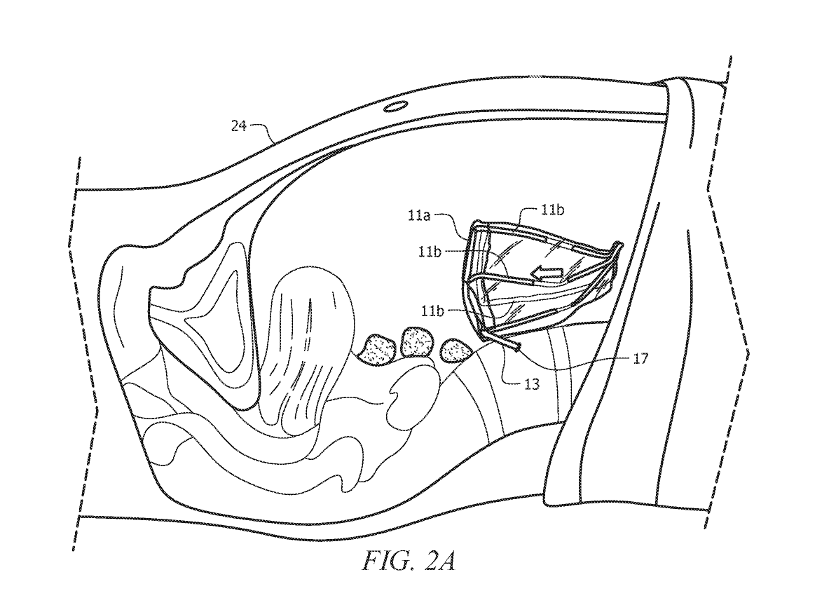

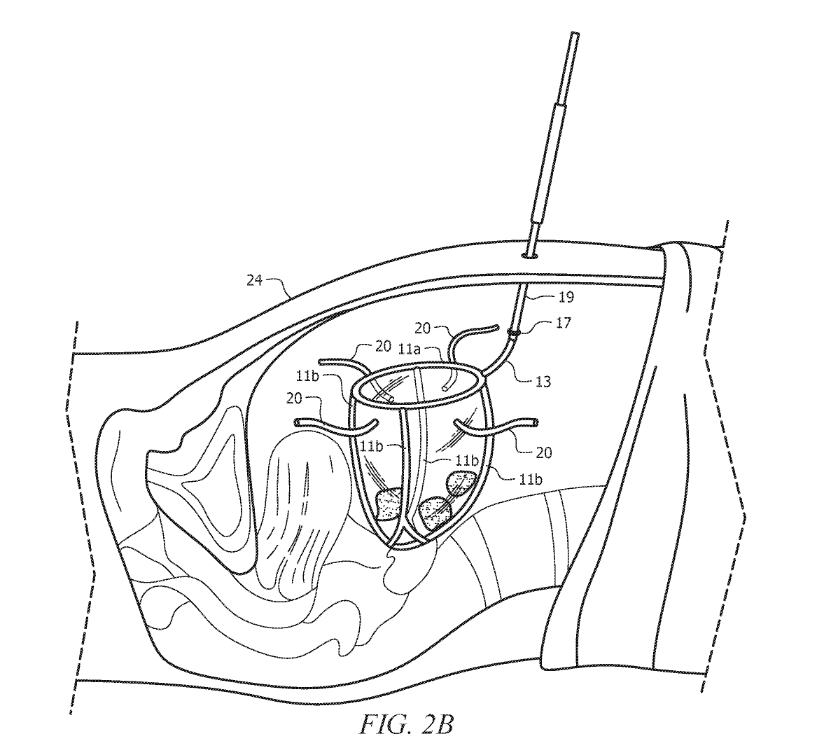

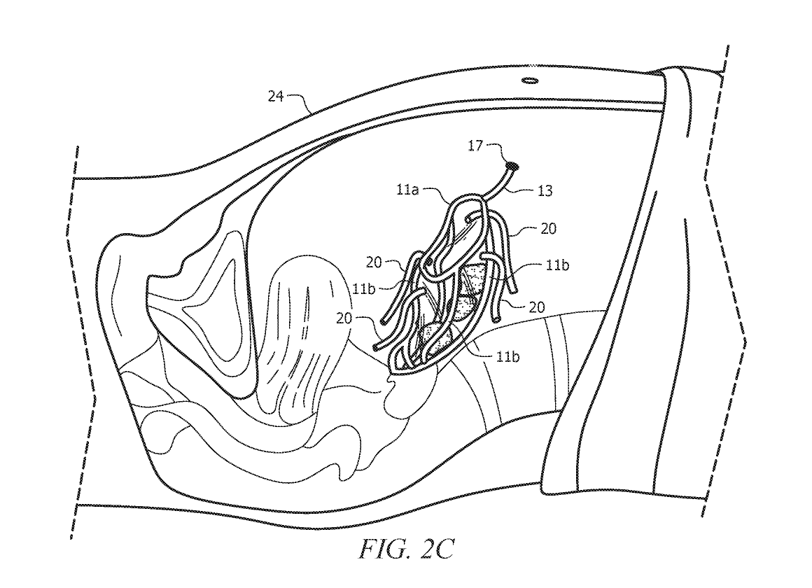

FIGS. 2A-C, 3A-3D, 4A-4B, 6, 6A-6D, and 7 depict an exemplary structure and methodology of the power morcellation system, apparatus, and method, including the pneumatic system as a novel component thereof, according to an embodiment of the current invention. The morcellation system is generally denoted by the reference numeral 10. It should be understood that, though the entirety of power morcellation system 10 is described herein, the current invention can be power morcellation system 10 or the pneumatic system by itself as may be applied within power morcellation system 10 or to a conventional endoscopic specimen containment bag/carrier. If the invention is considered to be the pneumatic system by itself, the remainder of this exemplary disclosure can be considered to simply show the system in use and how it may be used in such an endoscopic surgical procedure.

Structure

Structurally, as also seen in FIG. 1, power morcellation system 10 includes flexible retaining bag, pouch, or carrier 12 with large, typically circular or rounded specimen-receiving opening or aperture 14 that has a diameter/length and leads from the exterior of carrier 12 to substantially hollow interior 15 of carrier 12. A perimeter of opening 14 is lined with pneumatic channel 11a, and the outer sides of carrier 12 with a plurality of pneumatic channels 11b. At least one of pneumatic channels 11a, 11b includes filling channel 13 for coupling a conventional air or fluid source to channels 11a, 11b.

Optional valve 17 can be coupled anywhere along filling channel 13 in order to provide additional control over any air or fluid being pumped into channels 11a, 11b or withdrawn out of channels 11a, 11b. Any suitable control valve can be used as valve 17. Valve 17 would typically be positioned outside of body 24 of the patient or subject, such that user 30 has easier access to valve 17.

As previously discussed, this pneumatic system (substantially formed of pneumatic channel 11a, pneumatic channels 11b, filling channel 13, and optional valve 17 applied in a particular manner) can be used with any containment bag/carrier system having a specimen-receiving opening and a hollow interior as discussed. An example of such a containment bag/carder system is presented herein in order to fully illustrate the structure, function, and methodology of the pneumatic system, and as such, this exemplary containment bag/carder system is not intended to limit the scope of the underlying pneumatic system.

That being understood, the perimeter of specimen-receiving opening 14 can be lined with means of tightening, cinching or closing 16b carrier 12 or opening 14 by bringing all sides of the perimeter of opening 14 closer to one another, thus reducing or completely eliminating the diameter/length of specimen-receiving opening 14 as means of tightening, cinching, or closing 16a, 16b is actuated. Examples of means of tightening, cinching, or closing 16a, 16b include, but are not limited to, a drawstring-type apparatus that can be pulled relative to opening 14 to tighten or close opening 14, a monofilament suture that can be tied to cinch opening 14, a locking cable/zip tie-type apparatus that can be pulled relative to opening 14 to seal opening 14, and other known apparatuses and methods of tightening, cinching, or closing specimen-receiving opening 14. Through any of these means, specimen-receiving opening 14 can be tightened, cinched, or closed to hinder or prevent insufflating medium (gas) or any tissue inside carrier 12 from exiting interior 15 of carrier 12 through the specimen-receiving opening 14.

A plurality of flexible laparoscopic tool- or trocar-receiving channels, generally denoted by the reference numeral 20, extends externally from carrier 12 at a spaced distance away from specimen-receiving opening 14. Channels 20 have a proximal end that is closest to user 30 and may be free (i.e., not attached to anything at least initially), and also a distal end that terminates at carrier 12. Channels 20 are typically positioned between pneumatic channels 11b and extend from the sides of carrier 12 at a spaced distance away from pneumatic channel 11a. Channels 20 have a substantially hollow interior that is in open communication with interior 15 of carrier 12.

Typically, at least three (3) to four (4) channels are needed—1 for the morcellator, 1 for the control instrument, 1 for the camera, and 1 for insufflation. However, any number of port channels 20 are contemplated by the current invention. For example, one of the channels, such as the channel used for the camera, can also be utilized to link the insufflation source to substantially hollow interior 15 of carrier 12. Alternatively, another separate channel can be used for the insufflation source. Alternatively, even just one (1) laparoscopic port can be sufficient for the system 10, in particular if a camera is released or implanted within operative cavity 23 of the subject (see U.S. Pat. No. 8,416,342) and/or if a female subject's vagina is used as a laparoscopic port for the morcellator (see PCT App. No. PCT/US2013/050085) and/or if the control instrument and insufflation source use the same laparoscopic port using an integrated trocar, for example. One (1) laparoscopic port may even be suitable if using single-port laparoscopic techniques.

In an embodiment, conventional trocars (not shown) can be placed inside each channel 20. This can have the advantage of insufflating normally via an already-existing channel, such as the camera trocar insufflation point. An additional advantage of having a trocar inside each channel 20 is that it can facilitate manipulation of laparoscopic instrumentation without running the risk of perforating or otherwise damaging flexible channels 20.

Laparoscopic tool- or trocar-receiving channels 20 are positioned on and extend from carrier 12 so that channels 20 line up with the normal laparoscopic port placement (see reference numeral 26) on body 24 of the subject, with appropriate tolerances to allow for variations in placement of laparoscopic ports 26. Channels 20 are positioned at a spaced distance away from specimen-receiving opening 14 and at a spaced distance away from each other. Channels 26 typically are elongate and are structured to snugly fit various laparoscopic tools that can extend into interior 15 of carrier 12 via channels 26.

Each channel 20 may further include a long suture tag (not shown) at its open proximal end (opposite from its distal end terminating at retaining carrier 12) to facilitate laparoscopic manipulation, such as pulling the channel through the appropriate laparoscopic port 26.

When specimen-receiving opening 14 is tightened, cinched, or sealed via means 16a, 16b, excised tissue or specimen 22 can be sealed or otherwise contained within substantially interior 15 of carrier 12. Cinched opening 14 can be held to the anterior wall of the operative or targeted (e.g., abdominal, pelvic, peritoneal) cavity through the morcellator port and channel (or other existing laparoscopic port and channel) using a conventional laparoscopic tenaculum (not shown).

Power morcellation system/apparatus 10, in particular specimen-receiving carrier 12 and laparoscopic tool- or trocar-receiving channels 20 extending therefrom, is typically formed of pliable materials, such as flexible plastics, to permit entry and exit from one or more of laparoscopic ports 26 (e.g., removing carrier 12 and channels 20 through the morcellator port).

Methodology

The following steps are described in a manner and order that is not intended to be limiting of the scope of the current invention. It is contemplated herein that the order of the steps described herein can be altered or rearranged so long as the ultimate results of the steps remain the same or similar. This will become clearer as this specification continues.

As indicated previously, it should be noted as well that the current pneumatic system can be used by retrofitting onto an existing conventional containment bag/carrier system, or the pneumatic system can be formed and integrated with a conventional containment bag/carrier system. An example of such a containment bag/carrier system has been presented herein, and the methodology of performing a surgical procedure will be presented herein with this exemplary containment bag/carrier system in order to fully illustrate how the pneumatic system is used with a containment bag/carrier in the surgical procedure. This description of the methodology using this particular containment bag/carrier system is not intended to limit the scope of the underlying pneumatic system and use thereof in the surgical procedure.

That being understood, to begin, laparoscopic ports 26, typically at least three (3) or four (4) or more, are created in body 24 of the subject/patient according to conventional methods for surgical positioning of laparoscopic tools 20. If three (3) ports 24 are formed, ports 24 can function as a morcellator port (10-15 mm), a camera port, and a control instrument port. One of these ports can be used as the insufflation port as well, or a fourth port can be created for insufflation. Any number of laparoscopic ports 24 are contemplated by the current invention. For example, even just one (1) laparoscopic port can be sufficient for the system 10, in particular if a camera is released or implanted within operative cavity 23 of the subject (see U.S. Pat. No. 8,416,342) and/or if a female subject's vagina is used as a laparoscopic port for the morcellator (see PCT App. No. PCT/US2013/050085) and/or if the control instrument and insufflation source use the same laparoscopic port using an integrated trocar, for example. One (1) laparoscopic port may even be suitable if using single-port laparoscopic techniques.

Targeted tissue 22 (e.g., uterus, fibroid, etc.) is excised within interior or operative cavity 23 of body 24 using known endoscopic methods. Upon excising targeted tissue 22 using known methods (e.g., via the planned or intended morcellator port), the trocar is removed from the port (e.g., the planned or intended morcellator port), and the port skin incision can be enlarged as needed. At this point, laparoscopic ports 26 have been formed leading from the external environment to interior 23 (e.g., abdominal or pelvic cavity) of body 24, and targeted tissue 22 is contained within interior 23 (e.g., abdominal or pelvic cavity) of body 24.

Next, power morcellation system/apparatus 10—including the pneumatic system substantially formed of pneumatic channel 11a, pneumatic channels 11b, filling channel 13, and optional valve 17 applied in a particular manner—can be inserted into interior 23 of body 24 of the patient or subject via one of ports 26, typically the largest port, which may be the morcellator port. Flexible/Foldable retaining bag, pouch, or carrier 12 and laparoscopic tool- or trocar-receiving channels 20 extending from carrier 12 are passed through one of laparoscopic ports 26 into operative/peritoneal cavity 23, typically under camera visualization. Accordingly, the entirety of power morcellation system 10 is inserted into interior 23 of body 24 of the subject, primarily including pneumatic channels 11a, 11b, retaining carrier 12, and channels 20. This can be seen in FIG. 2A.

With channels 11a, 11b and carrier 12 disposed within body 24 of the subject, channels 11a, 11b are insufflated through filling channel 13 via a conventional air or fluid source outside body 24. Channels 11a, 11b are interconnected with one another, and their interiors are in open communication with one another. As such when filling channel 13 and/or valve 17 is coupled to the air or fluid source, fluid (e.g., gas, air) is pumped through filling channel 13 and into channels 11a, 11b, thus insufflating channels 11a, 11b and rigidifying carrier 12, as shown in FIG. 2B.

At this point or at a later time or even prior to this point), excised tissue or specimen 22 can then be placed into interior 15 of retaining carrier 12 through specimen-receiving opening 14 under camera visualization using graspers (such as grasper 28) inserted into operative cavity 23 via operating trocar ports 26. Having carrier 12 expanded or rigidified with large opening 14 not yet being cinched or closed facilitates placement of specimen 22 into interior 15 of carrier 12 through opening 14. FIG. 2B depicts specimen 22 within rigidified carrier 12.

With specimen 22 positioned within interior 15 of carrier 12, channels 11a, 11b may be desufflated prior to withdrawing channels 20 through ports 26. This can be an optional step. If channels 20 are withdrawn through ports 26 prior to insufflation of channels 11a, 11b and placement of specimen 22 within interior 15 of carrier 12, then carrier 12 may not be sufficiently flexible for easy placement of specimen 22 therein. If, on the other hand, channels 11a, 11b are not desufflated prior withdrawal of channels 20 through ports 26, then channels 20 may not fit through the trocars in ports 26. If channels 20 are forced through ports 26, one or more of channels 20 are at risk of breaking or being damaged. As such, it is contemplated herein that channels 11a, 11b are desufflated prior to withdrawing channels 20 through ports 26, as seen in FIG. 2C.

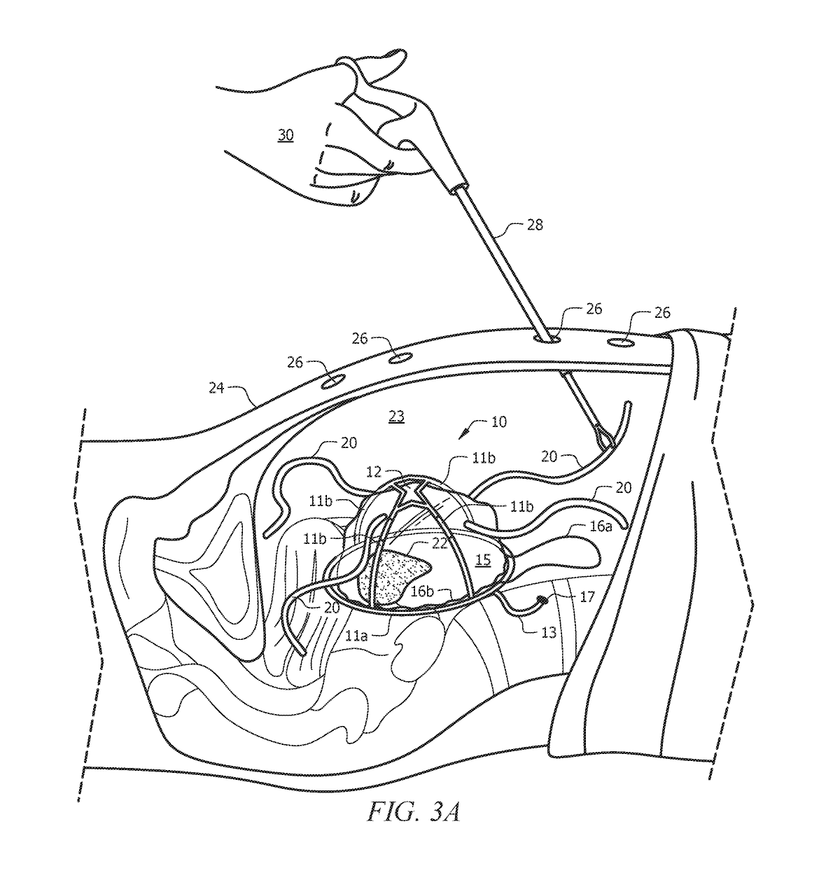

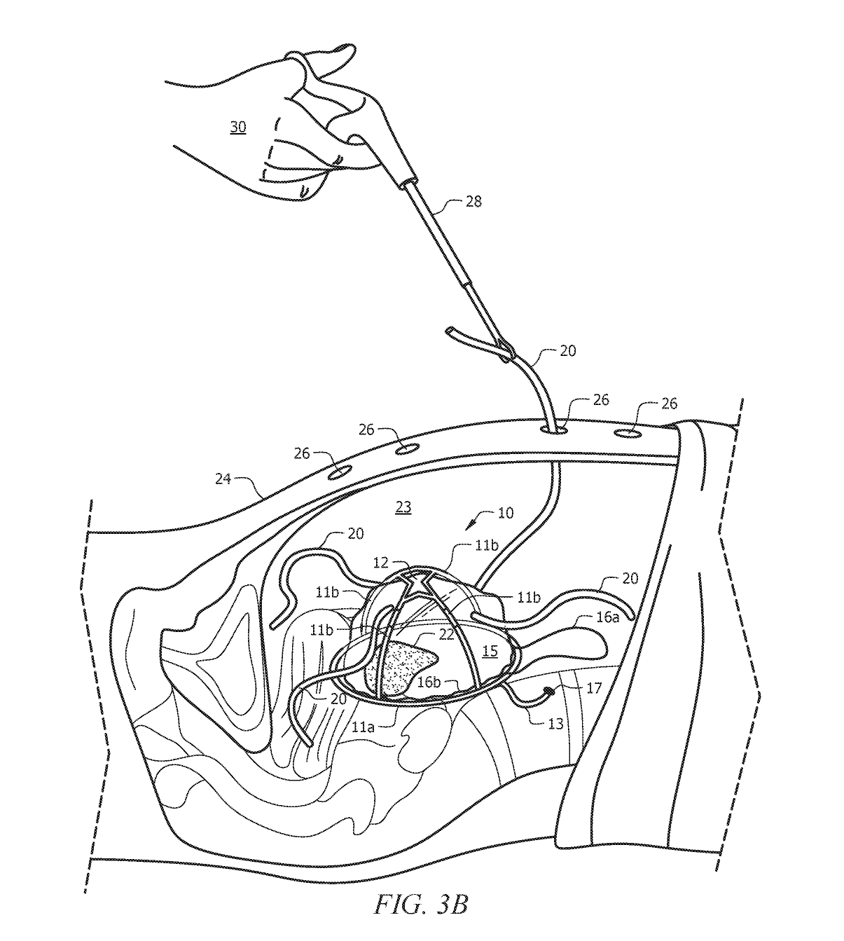

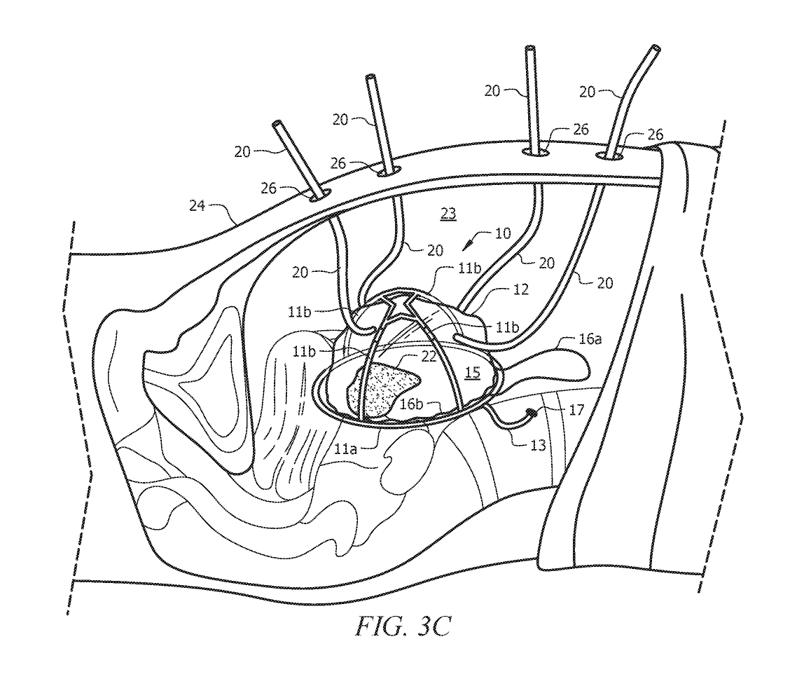

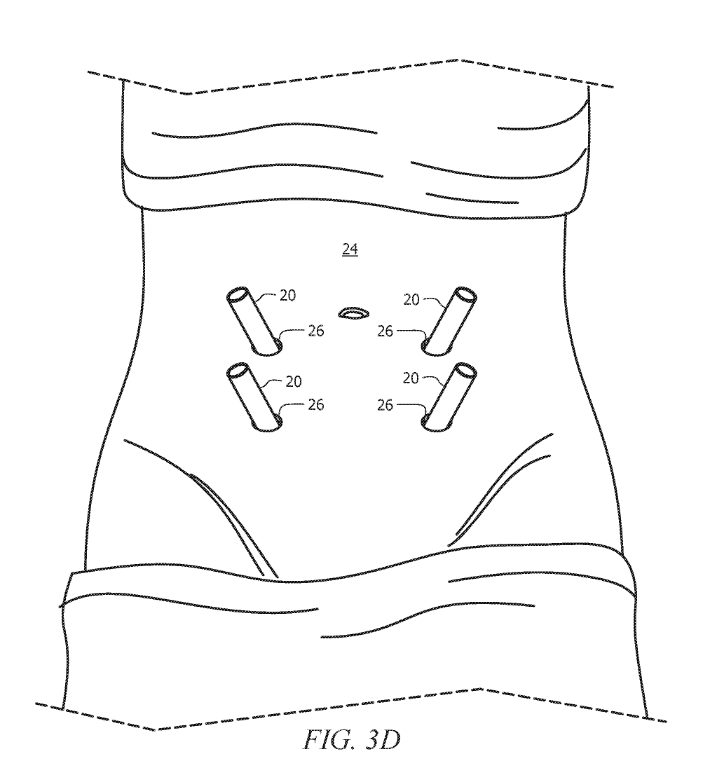

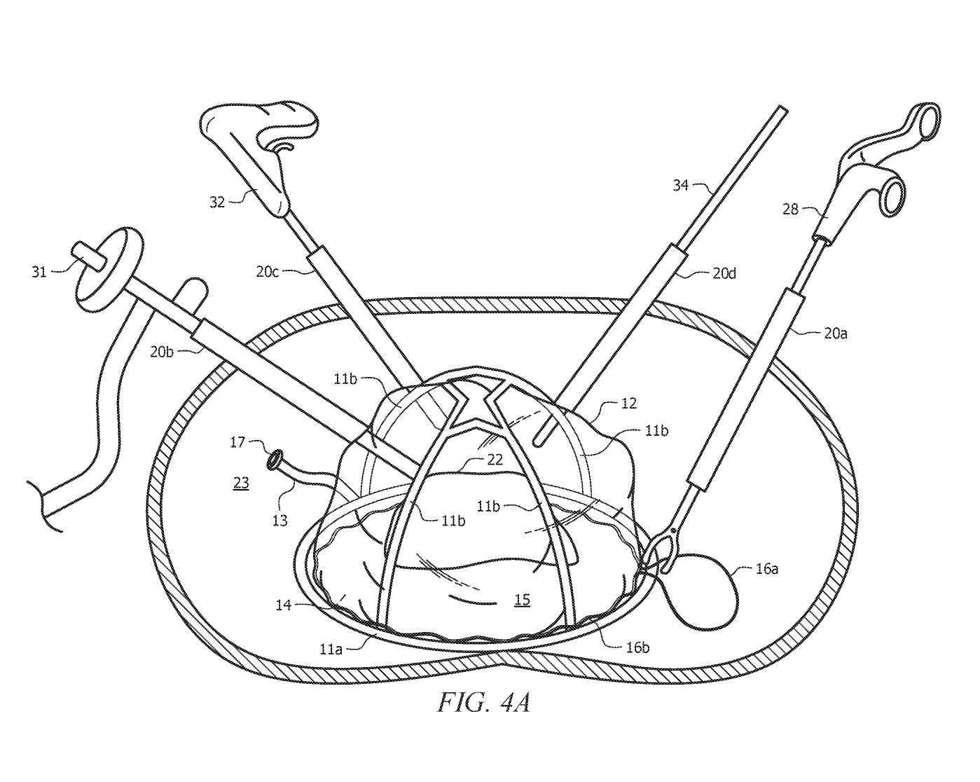

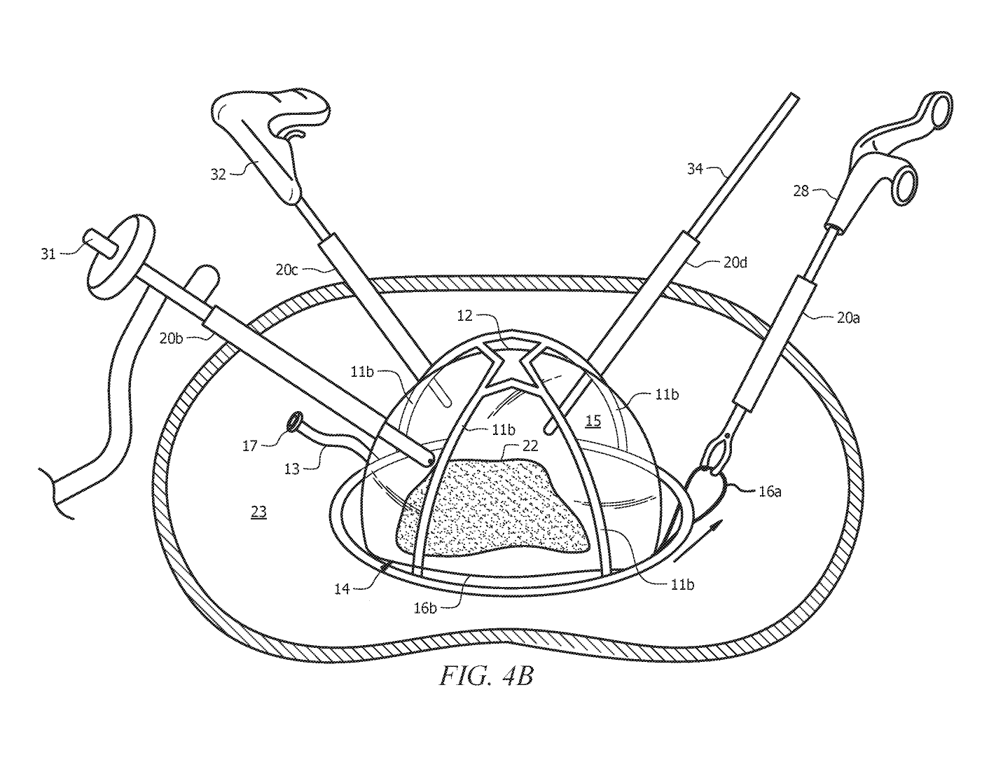

Now referring to FIG. 3A, with channels 11a, 11b desufflated and specimen 22 within interior 15 of carrier 12, grasper 28 can subsequently be inserted through one of ports 26 (e.g., the morcellator port or skin incision) by user 30. Grasper 28 can be used to withdraw one of channels 20 through one of ports 26, typically morcellator channel 30c (see FIGS. 4A-4B) through laparoscopic port 26 that was created for morcellator 32. Morcellator 32 itself can then be passed into retaining carrier 12 through morcellator channel 20c. If needed, morcellator channel 20c can be sealed around the morcellator trocar with a suture tie (not shown). Alternatively, control instrument channel 20a, camera channel 20b, or insufflation channel 20d can be withdrawn through their respective laparoscopic ports 26, rather than withdrawal of morcellator port 20c.

At this point, morcellator channel 20c has been pulled through its respective morcellator port 26, such that the free/proximal end of channel 20c is external to body 24 of the subject, and specimen 22 is positioned within interior 15 of carrier 12. Opening 14 of carrier 12 can then be cinched, closed, or sealed via means 16a, 16b. If a drawstring-type apparatus is used as means of closing 16a, 16b specimen-receiving opening 14, as seen in FIGS. 4A-4B, a suture retrieval needle (e.g., CARTER-THOMASON type) or other grasper (such as grasper 28 seen) can be passed transcutaneously through one of laparoscopic ports 26 to retrieve and/or pull the ends of the drawstring-type apparatus or other means 16a, 16b. This can be done to cinch opening 14, for example against the anterior abdominal wall, to form a relatively airtight seal and secure/clamp drawstring-type apparatus 16a in place within operative cavity 23. This can be seen in FIGS. 4A-4B, where FIG. 4A shows opening 14 in an open position and FIG. 4B shows opening 14 in a closed or cinched position. Alternatively, a knot can be tied in drawstring 16a, and opening 14 can be cinched with a knot pusher intracorporeally to ensure airtightness.

Alternatively, the specimen-receiving opening can be retrieved through one of the ports and tightened against the trocar to maintain pneumoperitoneum.

Though not required, the benefit of withdrawing channel 20c prior to actuating means 16a, 16b is for stability of system 10 during actuation of means 16a, 16b. For example, if means 16a, 16b is the drawstring shown in the figures, then channel 20c can be held outside of body 24 of the subject while drawstring 16a is pulled to tighten surrounding drawstring 16b. However, depending on which means 16a, 16b is used, this benefit of withdrawing channel 20c first may or may not be needed.

Alternatively, if a cable/zip tie is used as means of tightening or closing 16a, 16b specimen-receiving opening 14, opening 14 can be sealed by pulling the cable/zip tie with grasper 28 or a knot pusher. Any known means of tightening or closing 16a, 16b is contemplated herein by the current invention.

At this point or at any suitable time, the camera (seen in FIGS. 4A-4B as reference numeral 31), which may be used to visualize the positioning/withdrawal of morcellator channel 20c (or other channel 20) and the placement of excised specimen 22 into interior 15 of retaining carrier 12, can be removed from a central (typically camera) port, if that is where the camera was inserted, and placed in a side port/trocar. Similar to FIG. 3A, grasper 28 can subsequently be inserted through respective camera port or skin incision 26 (under direct visualization of camera 31) and used to withdraw camera channel 20b of morcellation system 10 through laparoscopic port 26 formed for camera 31. Camera 31 can then be removed from the side trocar port and itself passed into interior 15 of retaining carrier 12 through camera channel 20b.

At this point or beforehand, grasper 28 can subsequently be inserted through laparoscopic port or skin incision 26 formed for control instruments and used to withdraw control instrument channel 20a of morcellation system 10 through respective control instrument port 26. Control instrument (such as grasper 28; see FIG. 4B) itself can then be passed into interior 23 of retaining carrier 12 through control instrument channel 20a.

An insufflation tube can then be attached to the camera trocar or optionally passed through a separate insufflation channel which would be inserted and withdrawn as discussed with the previous channels 20. The insufflation tube would insufflate retaining carrier 12 to distend. If needed, channels 20 can be sealed against the trocars with suture ties.

More specifically, if a separate port is used for insufflation channel 20d, grasper 28 can be inserted through laparoscopic port or skin incision 26 formed for an insufflation source and used to withdraw insufflation channel 20d of morcellation system 10 through respective insufflation port 26. Insufflation channel 20d itself can then be passed into interior 15 of retaining carrier 12 through insufflation channel 20d. The proximal end of insufflation channel 20d (i.e., the end closes to user 30) can be coupled to a conventional insufflation source (not shown) for pumping fluid (e.g., air) into carrier 12 through insufflation channel 20d for insufflating carrier 12. As such, because opening 14 has been tightened, cinched, or closed via means 16a, 16b, the fluid supplied by the insufflation source should be not be able to escape interior 15 of carrier 23, or at the very least, the fluid should be hindered from exiting interior 15 of carrier 23 if opening 14 has been tightened but not closed completely. Insufflating carrier 12 forms a protected environment in which user 30 can morcellate and remove targeted tissue 22 or otherwise perform the necessary procedures.

Optionally, channels 11a, 11b can be insufflated as well to provide further expansion of carrier 12 or to further rigidify carrier 12 beyond insufflation of carrier 12 using insufflation channel 20d. Providing this additional expansion or rigidity may provide user 30 with a larger, more well-defined interior 15 of carrier 12 within which to morcellate or otherwise manipulate specimen 22.

FIGS. 3A-3D generally depict a process of withdrawing channels 20 from operative cavity 23 of a subject or patient after insertion of system 10 into body 24 of the subject or patient, placement of specimen 22 in interior 15 of carrier 12, and desufflation of channels 11a, 11b. FIG. 3A shows a grasper inserted through laparoscopic port 26 into operative cavity 23 of the patient or subject, where grasper 28 has grasped one of channels 20 within cavity 23. FIG. 3B shows grasper 28 retracting and withdrawing channel 20 through port 26. FIGS. 3C-3D show channels 20 fully withdrawn through ports 26 after grasper 28 has released channels 20. It is contemplated herein, however, that FIGS. 3A-3D show just one way of withdrawing channels 20 through laparoscopic ports 26; channels 20 can be withdrawn through their respective ports 26 using any contemplated methodology. FIGS. 3C-3D show channels 20 withdrawn through the respective laparoscopic ports 26 without any laparoscopic tools or trocars inserted into channels 20.

Regardless of the order of the foregoing steps of withdrawing channels 20 from operative cavity 23 and actuating means 16a, 16b to tighten or close opening 20, the ultimate goal is for channels 20 to be withdrawn from cavity 23 and for targeted specimen to be substantially sealed within interior 15 of carrier 12. As can be understood, this goal can be accomplished in a variety of manners, regardless of which of channels 20 are withdrawn first, when opening 20 is closed, etc.

Referring back to the exemplary methodology, at this point, channels 20a-20d have been pulled and withdrawn through laparoscopic ports 26, respectively, such that the free proximal end of each of channels 20a-20d are external to body 24 of the subject, and excised specimen 22 is sealed within interior 15 of carrier 12 after actuation of means 16a, 16b. As seen in FIG. 4B, control instrument 28, camera 31, morcellator 32, and insufflation source 34 extend into or are in communication with substantially hollow interior 15 of carrier 12. As discussed, for each channel 20a-20d that is withdrawn from body 24 of the subject through its respective port 26, a suture tag (not shown) can be attached to the free proximal end of each channel 20a-20d in order facilitate the withdrawal of channels 20a-20d from operative cavity 23 of the subject.

With these components of morcellation system 10 in place, morcellation of excised specimen/tissue 22 is performed under direct visualization of camera 31 (inserted through camera channel 20b), where fragments of specimen 22 can be morcellated and withdrawn by morcellator 32 through morcellator channel 20c and respective port 26. FIG. 5 shows a view from camera 31 within insufflated carrier 12, where control instrument 28 holds specimen 22 and morcellator 32 morcellates and removes specimen 22.

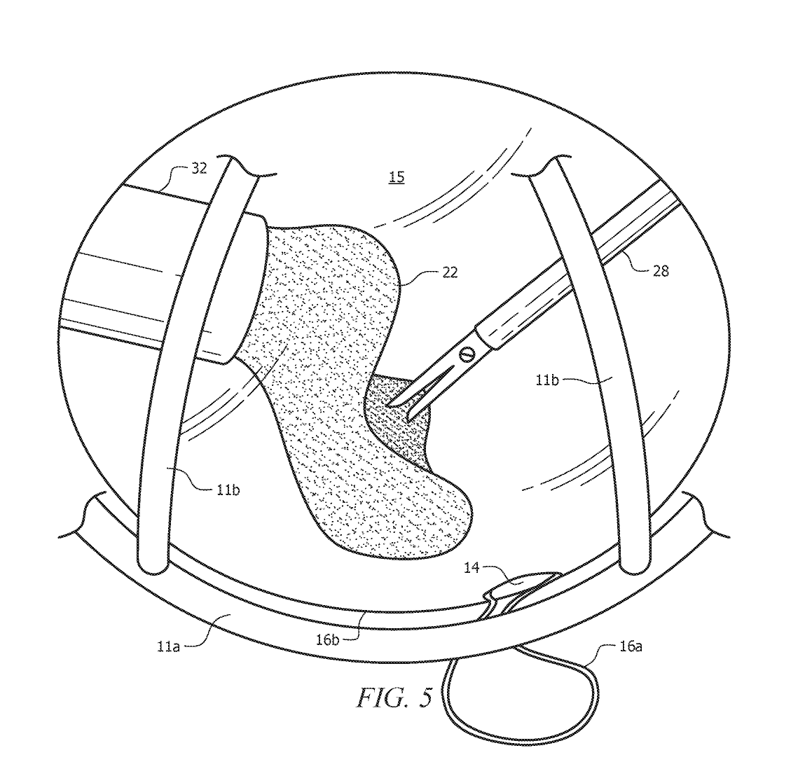

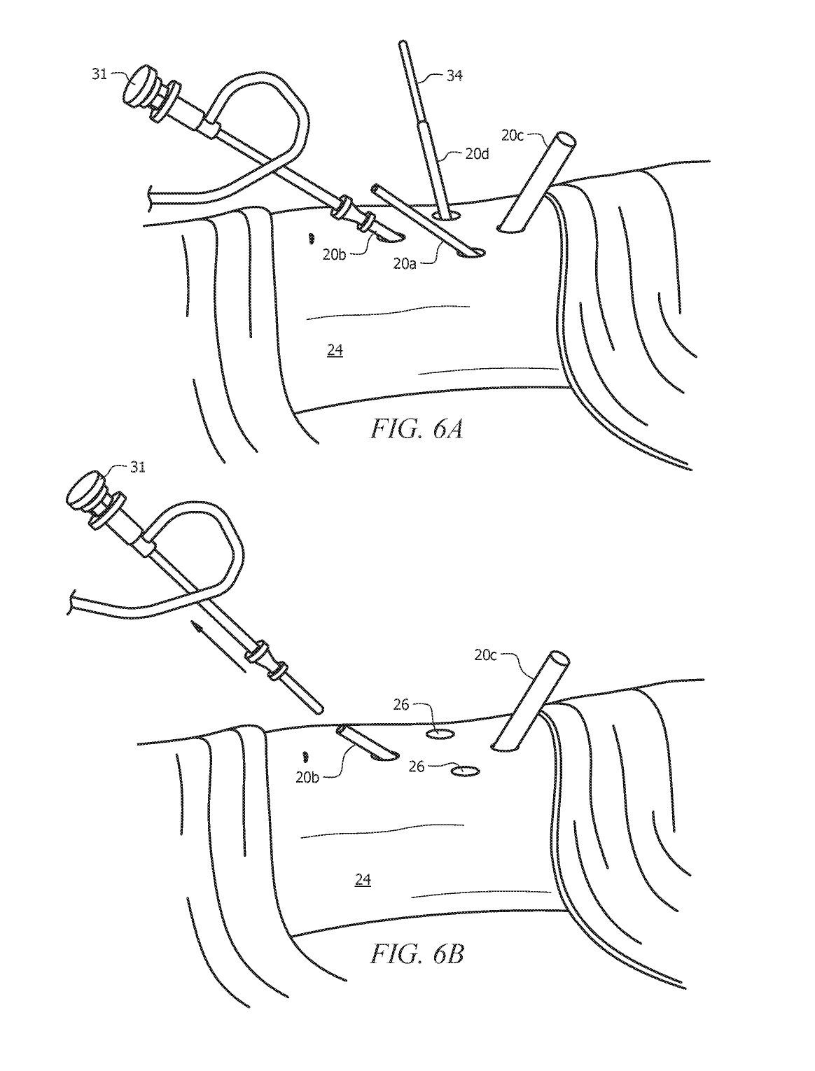

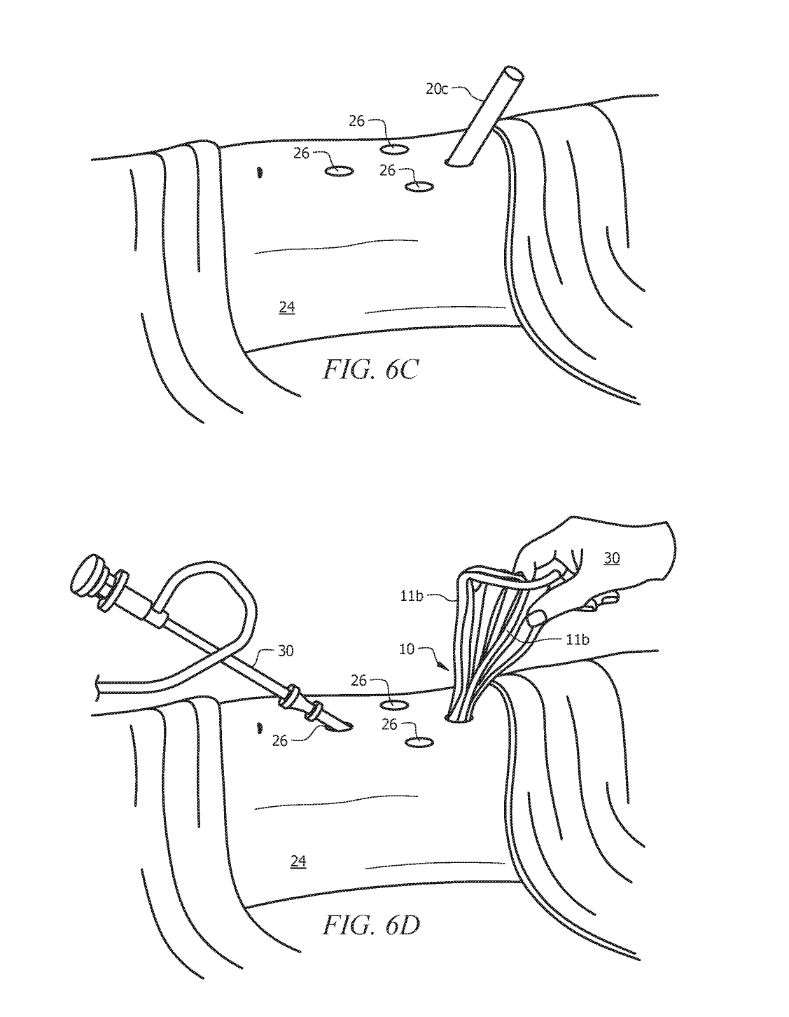

After morcellation is satisfactorily completed, retaining carrier 12 can be completely desufflated (via insufflation channel 20d and/or filling channel 13). Control instrument 28 and morcellator 32 can be withdrawn from channels 20a and 20c, respectively, with their respective trocars, leaving control instrument channel 20a and morcellation channel 20c extending externally from body 24 of the subject (FIG. 6A). The free/proximal end of control instrument channel 20c can be tied off, and channel 20c itself can be pushed back into operative cavity 23 of the subject. Removal of insufflation source 34 and insufflation port 20d can occur in substantially a similar manner. See FIG. 6B.

Camera 31 may remain in camera channel 20b through this process for the purpose of visualization, but beforehand or afterwards, camera 31 can be withdrawn from camera channel 20b with its respective trocar, leaving camera channel 20b extending externally from body 24 of the subject (FIG. 6B). The free/proximal end of camera channel 20b can be tied off, and channel 20b itself can be pushed back into operative cavity 23 of the subject. See FIG. 6C.

Morcellator 32 can be removed, if not previously removed, and subsequently, the intact, desufflated retaining carrier 12, along with pneumatic channels 11a, 11b, control instrument channel 20a, camera channel 20b, and camera channel 20c—all of which are within body 24 of the subject—can be withdrawn via laparoscopic port 26 that was formed for morcellator 32 (assuming the morcellator port is the largest in size). To do this, as can be seen in FIG. 6D, user 30 can simply roll or fold carrier 12 (channels 11a, 11b can be used to facilitate this rolling/folding) and pull morcellator channel 20c to withdraw pliable carrier 12, channels 11a, 11b, and pliable channels 20a-20d from interior 23 of body 24 of the subject. Though typically morcellator channel 20c is largest in size/diameter, it is contemplated herein that removal of system 10 can occur through any suitable port 26.

Regardless of the order of the foregoing steps of removing the laparoscopic tools from channels 20 or how cavity 23 is visualized or which of channels 20 are inserted back into cavity 23, the ultimate goal is for system 10 to be entirely withdrawn from cavity 23 through at least one of laparoscopic ports 24. As can be understood, this goal can be accomplished in a variety of manners, regardless of which of tools are removed first, which of channels 20 are pushed into cavity 23, which of ports 24 is used for withdrawing system 10, etc.

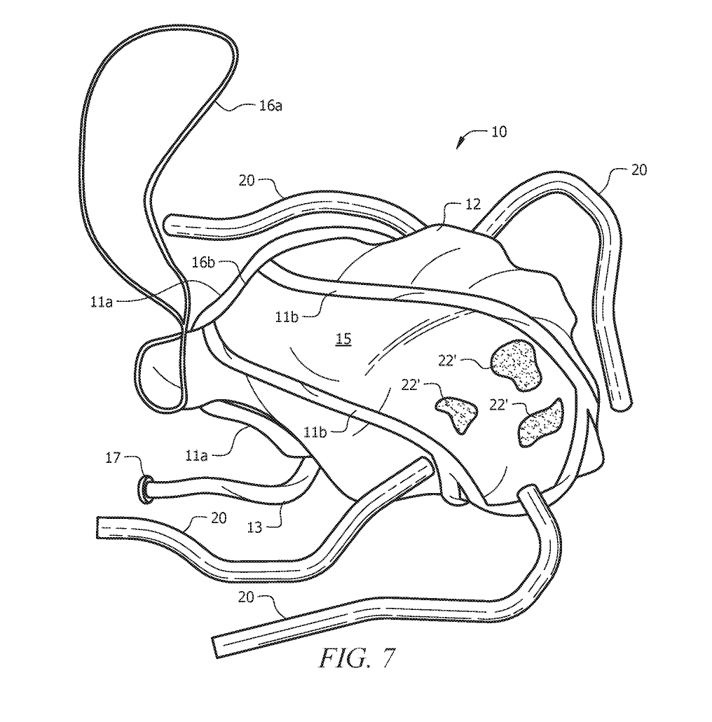

As can be seen in FIG. 7, remnants 22′ of specimen 22 that were not withdrawn via morcellator 32 remain contained within retaining carrier 12 throughout the morcellation procedure and even after withdrawal from body 24 of the subject.

At this point, all laparoscopic instruments can be cleaned and replaced in the laparoscopic ports, as necessary, to inspect operative/peritoneal cavity 23. When satisfied, the port fascia can be closed using known methods. The remaining procedure can be performed using known methods as well.

Glossary of Claim Terms

Apex: This term is used herein to refer to a point on the retaining carrier that is substantially central and opposite from the specimen-receiving opening. It would be from this point that the pneumatic side channels can extend radially outwardly along the sides of the carrier. It would also be the point at which the pneumatic side channels can converge/intersect so that the interiors of each of the pneumatic side channels are in open communication with each other.

Applying a force: This term is used herein to refer to an act of manipulating a structure to act in a manner desired. As an example of the current invention, a pulling force can be applied to a withdrawn channel in order to extract the entire morcellation system (and components thereof, i.e., retaining carrier, other channels, etc.) from the inside of a patient or subject.

Control instrument: This term is used herein to refer to any laparoscopic tool that can be used in holding or stabilizing a specimen or tissue during operation of the surgical procedure, for example during morcellation of the specimen or tissue.

Desufflate: This term is used herein to refer to a fluid (e.g., gas) exiting a wholly or partially inflated reservoir or carrier such that the reservoir or carrier is no longer inflated.

Direct visualization: This term is used herein to refer to the ability of a user or operator (e.g., surgeon) to consistently see or recognize the procedure being performed within the interior of the subject or patient.

Distal: This term is used herein to refer to a position of a structure that is closer to the interior of a subject or patient than another structure that is closer to a user or operator (e.g., surgeon).

Drawstring-type apparatus: This term is used herein to refer to a string, cord, or similar structure lining the perimeter of an opening and laced through eyelets for use in tightening, cinching, closing, or sealing off the opening.

Excised specimen: This term is used herein to refer to tissue within a subject or patient intended to undergo a medical procedure, for example morcellation and removal from the subject or patient.

Filling channel: This term is used herein to refer to a medium that is, on one end, directly or indirectly coupled to a fluid source that pumps or otherwise provides a fluid to flow therethrough. On an opposite end, the filling channel is coupled to one or more of the pneumatic channels disposed on the retaining carrier.

Fluid source: This term is used herein to refer to any controllable supply of a gas or liquid. An example is an air pump.

Fluid: This term is used herein to refer to any substance that can flow smoothly. The fluid can be a gas (e.g., air) or a liquid.

Insufflate: This term is used herein to refer to pumping a fluid (e.g., gas) into the interior of a reservoir or carrier in order to inflate the reservoir or carrier, thus providing a substantially open space for conducting the medical procedure at hand.

Interconnected: This term is used herein to refer to components, such as pneumatic channels, being linked to each other, such that their respective interiors are in communication with each other.

Laparoscopic port: This term is used herein to refer to an incision or aperture in the skin or body of a subject or patient that leads from an environment external to the body of the subject to an environment internal to the body of the subject. It is contemplated herein that a laparoscopic port can, for example, be an incision leading to the peritoneal cavity of the subject or even be a vagina of a female subject.

Laparoscopic tool: This term is used herein to refer to a surgical instrument that can be used during minimally invasive surgery, where the laparoscopic tool can be inserted through a laparoscopic port.

Means of tightening, cinching, closing, or sealing: This term is used herein to refer to any suitable apparatus or methodology of enclosing a targeted specimen/tissue within a carrier, such that the interior of the carrier is not in completely open communication with an environment external to the carrier.

Operative internal cavity: This term is used herein to refer to space within a subject or patient where a medical procedure is intended to take place. Examples of operative internal cavities include, but are not limited to, peritoneal cavities, abdominal cavities, and pelvic cavities.

Pneumatic channel: This term is used herein to refer to an elongate medium through which a fluid, such as air, can flow. When such a channel is filled with the fluid, the channel can be understood to become more rigid.

Proximal: This term is used herein to refer to a position of a structure that is closer to a user or operator (e.g., surgeon) than another structure that is closer to the interior of a subject or patient.

Retaining carrier: This term is used herein to refer to a resilient bag or pouch that has an opening for receiving a specimen/tissue, where the bag or pouch can enclose the specimen/tissue and create an environment for performing a medical procedure on the specimen/tissue.

Rigidify: This term is used herein to refer to causing something to become stiffer or more fixed than prior to being rigidified. It can be understood that when the pneumatic channels discussed herein are filled with a fluid, such as air, they inflate and expand outwardly, thus stretching the pliable material of the retaining carrier. In turn, this causes both the pneumatic channels and the containment carrier to become stiffer or more fixed than prior to the pneumatic channels being filled with the fluid.

Substantially equidistant: This term is used herein to refer to the pneumatic channels having positions along the sides of the retaining carrier, such that they have a substantially equal distance from each other around the carrier. For example, if there are four (4) pneumatic side channels, each would occupy a substantially central position in each quadrant around the carrier. It should be noted that it is not imperative that they are exactly equally distant from each other, but a substantially equal distance between adjacent channels facilitates an even expansion of the carrier.

Substantially hollow: This term is used herein to refer to a space or cavity that is sufficiently empty to permit an intended action therewithin. For example, the retaining carrier has a “substantially hollow” interior, meaning it needs to have sufficient empty space to fit the laparoscopic tools used, perform morcellation of the excised specimen, withdraw the specimen, etc. If other items are disposed within the retaining carrier's interior, that is acceptable as long as the intended actions can still be performed effectively.

Substantially opposite: This term is used herein to refer to a convergence/intersection point of the pneumatic side channels being located across the retaining carrier from a center of the specimen-receiving opening. It should be noted that it is not imperative that the convergence/intersection is exactly directly across from the center of the specimen-receiving opening, but just close enough to facilitate a symmetrical and outward expansion of the retaining carrier.

Surface: This term is used herein to refer to a layer of material that lines the interior or exterior of a bag, pouch, or carrier.

Suture tag: This term is used herein to refer to an apparatus connected to a channel according to the current invention and use for the purpose of facilitating manipulation of the channel (e.g., withdrawing the channel through the laparoscopic port).

Valve: This term is used herein to refer to any device or component that controls the flow of a fluid (gas or liquid) through a channel or medium.

Withdraw: This term is used herein to refer to extracting an object or component from the interior of a body of a subject or patient and pulling/bringing it to the exterior of the body of the subject or patient (e.g., through a laparoscopic port). The object or component can be withdrawn partially or fully. For example, a channel can be withdrawn such that a portion (typically a majority) of the channel is positioned outside of the body and a portion of the channel is still positioned inside of the body. As another example, a morcellation system can be withdrawn from the interior of the body by extracting the entire apparatus from the interior of the body, such that no remaining portion of the system remains inside the body.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

What is claimed is:

1. A pneumatic system for use with a pliable endoscopic retaining carrier having a specimen-receiving opening and a substantially hollow interior, where said retaining carrier is insertable into an operative internal cavity of a subject or patient, said pneumatic system comprising:
a pneumatic base channel disposed along a perimeter of said specimen-receiving opening of said retaining carrier;
a plurality of pneumatic side channels disposed at a spaced distance away from each other along an outer surface of a side of said retaining carrier,
wherein said pneumatic base channel and said plurality of side channels collectively form pneumatic channels of said pneumatic system,
wherein said pneumatic channels are interconnected and have substantially hollow interiors that are in open communication with each other; and
a filling channel in communication with said pneumatic channels, said filling channel extending from a pneumatic channel selected from said pneumatic channels,
said filling channel having an end configured to be coupled to a fluid source for pumping a fluid into said filling channel and thus also into said interiors of said pneumatic channels,
wherein said pneumatic channels each have a desufflated position and an insufflated position, and wherein said retaining carrier has a collapsed position and a rigidified position,
said desufflated position of said pneumatic channels corresponding to said collapsed position of said retaining carrier used during insertion and withdrawal of said retaining carrier, including said pneumatic channels, into and out of said operative internal cavity of said subject or patient,
said insufflated position of said pneumatic channels corresponding to said rigidified position of said retaining carrier used during placement of an excised specimen within said subject's internal operative cavity into said substantially hollow interior of said retaining carrier,
wherein insufflation of said pneumatic channels rigidifies said retaining carrier and desufflation of said pneumatic channels collapses said retaining carrier.

2. A pneumatic system as in claim 1, further comprising:
a valve disposed on and in communication with said filling channel, said valve configured to be coupled to said fluid source for controlling a flow of said fluid into said filling channel and into said pneumatic channels.

3. A pneumatic system as in claim 1, wherein said filling channel is in direct communication with an interior of said pneumatic base channel.

4. A pneumatic system as in claim 1, wherein said plurality of pneumatic side channels are disposed substantially equidistant from each other around said retaining carrier.

5. A pneumatic system as in claim 4, wherein said plurality of pneumatic side channels extend along a length of said retaining carrier and converge at an apex of said retaining carrier, said apex being on a substantially opposite side of said retaining carrier from said specimen-receiving opening.

6. An endoscopic apparatus for retrieving an excised specimen within an operative internal cavity of a subject or patient, comprising:
a pliable retaining carrier having a specimen-receiving opening and a substantially hollow interior, said retaining carrier insertable into said operative internal cavity of said subject or patient;
a pneumatic base channel disposed along a perimeter of said specimen receiving opening of said retaining carrier;
a plurality of pneumatic side channels disposed at a spaced distance away from each other along an outer surface of a side of said retaining carrier,
wherein said pneumatic base channel and said plurality of side channels collectively form pneumatic channels of said pneumatic system,
wherein said pneumatic channels are interconnected and have substantially hollow interiors that are in open communication with each other; and
a filling channel in communication with said pneumatic channels, said filling channel extending from a pneumatic channel selected from said pneumatic channels,
said filling channel having an end configured to be coupled to a fluid source for pumping a fluid into said filling channel and thus also into said interiors of said pneumatic channels,
wherein said pneumatic channels each have a desufflated position and an insufflated position, and wherein said retaining carrier has a collapsed position and a rigidified position,
said desufflated position of said pneumatic channels corresponding to said collapsed position of said retaining carrier used during insertion and withdrawal of said retaining carrier, including said pneumatic channels, into and out of said internal operative cavity of said subject or patient,
said insufflated position of said pneumatic channels corresponding to said rigidified position of said retaining carrier used during placement of said excised specimen within said subject's internal operative cavity into said substantially hollow interior of said retaining carrier,
wherein insufflation of said pneumatic channels rigidifies said retaining carrier and desufflation of said pneumatic channels collapses said retaining carrier.

7. An endoscopic apparatus as in claim 6, further comprising a valve disposed on and in communication with said filling channel, said valve configured to be coupled to said fluid source for controlling a flow of said fluid into said filling channel and into said pneumatic channels.

8. An endoscopic apparatus as in claim 6, wherein said filling channel is in direct communication with an interior of said pneumatic base channel.

9. An endoscopic apparatus in claim 6, wherein said plurality of pneumatic side channels are disposed substantially equidistant from each other around said retaining carrier.

10. An endoscopic apparatus as in claim 9, wherein said plurality of pneumatic side channels extend along a length of said retaining carrier and converge at an apex of said retaining carrier, said apex being on a substantially opposite side of said retaining carrier from said specimen-receiving opening.

11. An endoscopic apparatus as in claim 6, further comprising:
a means of tightening, cinching, closing, or sealing said specimen-receiving opening positioned along said perimeter of said specimen-receiving opening.

12. A morcellation system as in claim 11, further comprising:
said means of tightening, cinching, closing, or sealing said specimen-receiving opening being a drawstring-type apparatus that is pulled relative to said specimen-receiving opening in order to reduce a diameter or length of said specimen-receiving opening.

13. An endoscopic apparatus as in claim 11, further comprising:
a plurality of elongate, flexible laparoscopic tool- or trocar-receiving channels extending externally from said outer surface of said side of said retaining carrier at a spaced distance away from said specimen-receiving opening, at a spaced distance away from each other, and at a spaced distance away from said pneumatic channels,
said plurality of channels positioned on said outer surface of said retaining carrier such that said plurality of channels line up with laparoscopic ports on a body of said subject,
said plurality of channels structured to receive one or more laparoscopic tools,
said plurality of channels each having a proximal end and a distal end, said distal end of said each channel terminating at said side of said retaining carrier within said operative internal cavity of said subject, said proximal end being external to said body of said subject when said morcellation system has been inserted into said patient or subject,
said each channel having a substantially hollow interior that is in communication with said substantially hollow interior of said retaining carrier,
said retaining carrier further having an insufflated position within said abdominal or pelvic cavity of said subject with said specimen-receiving opening being closed or cinched.

14. An endoscopic apparatus as in claim 13, further comprising:
said operative internal cavity of said subject or patient being an abdominal or pelvic cavity.

15. An endoscopic apparatus as in claim 13, further comprising:
said one or more laparoscopic tools selected from the group consisting of a trocar, a morcellator, a camera, a control instrument, and an insufflation source.

16. An endoscopic apparatus as in claim 13, further comprising:
said plurality of channels including a morcellator channel structured to receive a morcellator, a control instrument channel structured to receive a control instrument, and a camera channel structured to receive a camera, so that said morcellator can morcellate said targeted specimen within said substantially hollow interior of said retaining carrier under direct visualization of said camera while said control instrument holds said targeted specimen.

17. An endoscopic apparatus as in claim 13, further comprising:
an elongate suture tag attached to and positioned at said second end of said each channel to facilitate laparoscopic manipulation of said each channel.

18. An endoscopic apparatus as in claim 13, further comprising:
said insufflated position of said retaining carrier further including said closed or cinched specimen-receiving opening being pressed against an anterior abdominal wall within said subject when said operative internal cavity is an abdominal or pelvic cavity of said subject.

19. A method of performing minimally invasive laparoscopic surgery on a subject or patient, comprising:
providing a plurality of laparoscopic ports in a body of said subject or patient;
excising a targeted specimen within an operative internal cavity of said subject;
inserting a pneumatic morcellation system in a deflated position into said operative internal cavity of said subject or patient, said pneumatic morcellation system including a pliable retaining carrier having a substantially hollow interior and a closeable or sealable specimen-receiving opening, said specimen-receiving opening providing for completely open communication between said operative internal cavity of said subject or patient and said substantially hollow interior of said retaining carrier,
said pneumatic morcellation system further including a pneumatic base channel disposed along a perimeter of said specimen-receiving opening, a plurality of pneumatic side channels disposed along an outer surface of said retaining carrier, and a filling channel in communication with said pneumatic base channel and said pneumatic side channels;
insufflating said pneumatic base channel and said pneumatic side channels through said filling channel to expand or rigidify said retaining carrier; and
positioning said excised, targeted specimen within said substantially hollow interior of said retaining carrier through said specimen-receiving opening.

20. A method as in claim 19, further comprising:
prior to the step of inserting said pneumatic morcellation system into said operative internal cavity of said subject or patient, attaching said pneumatic base channel along said perimeter of said specimen-receiving opening of said retaining carrier and attaching said pneumatic side channels along said outer surface of said retaining carrier.

21. A method as in claim 19, further comprising:
said operative internal cavity of said subject or patient being an abdominal or pelvic cavity.

22. A method as in claim 21, further comprising:
said excised, targeted specimen being a uterus in a female subject or patient.

23. A method as in claim 19, further comprising:
said pneumatic morcellation system further including a plurality of laparoscopic tool- or trocar-receiving channels extending from said outer surface of said retaining carrier, said plurality of channels having an interior being in open communication with said substantially hollow interior of said retaining carrier, said plurality of channels positioned at a spaced distance away from said specimen-receiving opening and at a spaced distance away from each other, said plurality of channels being spatially aligned with said plurality of laparoscopic ports, said each channel having a first end that terminates at said outer surface of said retaining carrier within said operative internal cavity of said subject or patient and a second end that is external to said body of said subject or patient when said pneumatic morcellation system has been inserted into said patient or subject or patient;
withdrawing each of said plurality of channels from said operative internal cavity through a respective spatially aligned laparoscopic port of said plurality of laparoscopic ports;
tightening, cinching, closing, or sealing said specimen-receiving opening to enclose said excised, targeted specimen within said substantially hollow interior of said retaining carrier, such that said substantially hollow interior of said retaining carrier is not in completely open communication with said operative internal cavity of said subject or patient;
inserting one or more laparoscopic tools into at least one of said plurality of channels, wherein said laparoscopic tool has a distal end disposed within said substantially hollow interior of said retaining carrier and a proximal end disposed external to said body of said subject or patient;
insufflating said retaining carrier to distend in order to form a protected environment;
morcellating said excised, targeted specimen within said insufflated retaining carrier in order to remove at least a majority of said excised, targeted specimen from said operative internal cavity of said subject or patient;
desufflating said retaining carrier; and
withdrawing said pneumatic morcellation system including said retaining carrier and said plurality of channels from said operative internal cavity through a laparoscopic port of said plurality of laparoscopic ports, said retaining carrier enclosing any remnants of said excised, targeted specimen in said substantially hollow interior.

24. A method as in claim 23, further comprising:
prior to the step of withdrawing said each channel from said operative internal cavity of said subject or patient, desufflating said pneumatic base channel and said pneumatic side channels.

25. A method as in claim 24, further comprising:
after the step of withdrawing said each channel from said operative internal cavity of said subject or patient, re-insufflating said pneumatic base channel and said pneumatic side channels to further rigidify said retaining carrier to form said protected environment.

26. A method as in claim 23, further comprising step of:
positioning said tightened, cinched, closed, or sealed specimen-receiving opening against an anterior abdominal or pelvic wall within said subject or patient when said operative internal cavity is an abdominal or pelvic cavity of said subject or patient.

27. A method as in claim 23, further comprising:
said step of withdrawing said each channel from said operative internal cavity performed by:
inserting a grasper into a first laparoscopic port of said plurality of laparoscopic ports and withdrawing a first channel of said plurality of channels through said first laparoscopic port, and
inserting said grasper into a second laparoscopic port of said plurality of laparoscopic ports and withdrawing a second channel of said plurality of channels through said second laparoscopic port,
wherein said step of tightening, cinching, closing, or sealing said specimen-receiving opening is performed after withdrawing said first channel but before withdrawing said second channel.

28. A method as in claim 23, further comprising:
said step of withdrawing said pneumatic morcellation system from said operative internal cavity performed by:
inserting said each channel into said operative internal cavity except for one (1) of said plurality of channels, and
applying a force to said one channel in order to extract said retaining carrier and said each channel from said operative internal cavity.

29. A method as in claim 23, further comprising:
performing each of the steps of said method of performing minimally invasive laparoscopic surgery on said subject or patient under direct visualization of a camera.
